(19) United States
(12) Patent Application Publication
(10) Pub. No.: US 2023/0329785 A1
(43) Pub. Date: Oct. 19, 2023

(54) ENDOSCOPIC COHERENT TISSUE ABLATION

(71) Applicant: GlaiveRF Inc., Carlisle, MA (US)

(72) Inventors: Wayne H. Woods, JR., Carlisle, MA (US); Christopher Shelby, Shreveport, LA (US)

(21) Appl. No.: 18/134,589

(22) Filed: Apr. 14, 2023

Related U.S. Application Data
(60) Provisional application No. 63/331,511, filed on Apr. 15, 2022.
(60) Provisional application No. 63/355,162, filed on Jun. 24, 2022.

(51) Int. Cl. A61B 18/20; A61B 1/00; A61B 1/05; A61B 1/04; A61B 18/00; A61B 17/00; A61F 9/008

(52) U.S. Cl. CPC A61B 18/20; A61B 1/000094; A61B 1/051; A61B 1/000096; A61B 1/041; A61B 2018/00982; A61B 2017/00296; A61B 2018/2035; A61B 2018/00904; A61F 2009/00868; A61B 2018/00577; A61F 9/00802

(57) ABSTRACT

Devices and methods for endoscopic coherent tissue ablation are provided. An endoscopic ablation system includes: an endoscope; an ablation optical phased array (AOPA) with the endoscope, the AOPA being configured to emit a beam in a selectively controllable direction, the beam being configured to ablate tissue; and an imaging system with the endoscope, the imaging system configured to capture images in the vicinity of the endoscope and/or the AOPA.

### RELATED APPLICATIONS

This application claims priority to U.S. provisional application No. 63/331,511 filed Apr. 15, 2022, and U.S. provisional application No. 63/355,162 filed Jun. 24, 2022, both of which are incorporated by reference herein in their entirety.

### BACKGROUND

The present invention relates generally to medical devices and methods of use and, more particularly, to devices and methods for endoscopic coherent tissue ablation.

Ablation is a medical term that means removal or eradication of tissue. Endoscopic ablation therapy is commonly performed for a precancerous condition called Barrett's Esophagus. Ablation therapy is a minimally invasive procedure that removes diseased cells in the mucosal layer of the esophagus. The removal is done by means of an endoscope and a treatment modality such as cryotherapy, photodynamic therapy, or radiofrequency ablation.

The type of treatment chosen for patients is determined on a case-by-case basis, taking into consideration several factors, including how long the Barrett's segment is within the esophagus, the patient's own symptoms, and his or her capability for follow-up treatment. Patients undergoing treatment will also continue lifestyle changes and drug therapy for reflux (GERD) disease.

Cryotherapy—also referred to as cryo-ablation, cryosurgery or cryospray—is the use of extreme cold to destroy diseased tissue. Cryotherapy is often chosen by gastroenterologists because it allows them to reach difficult-to-treat areas within the esophageal lining. It is a relatively quick outpatient procedure for patients and has few side effects.

During the procedure, patients are first given a mild sedative. The gastroenterologist then gently maneuvers an endoscope down the esophagus. The endoscope contains a catheter and a miniature camera, which allows the gastroenterologist to view images of the diseased area on a video monitor.

Once the treatment area is identified, the gastroenterologist sprays liquid nitrogen or argon gas through the catheter at a low pressure onto the segment of the esophageal lining that has Barrett's esophagus. The frozen tissue is allowed to thaw and then is sprayed again. The treatment freezes and kills the diseased cells, allowing regeneration of new healthy cells. The procedure takes about 20 to 30 minutes. Several treatments over several months may be performed.

After the procedure, patients may experience minor swallowing difficulties for a few days. However, most patients are able to go back to work the following day. Patients have a regular follow-up visit with their gastroenterologist after the procedure.

Photodynamic therapy is the use of laser light in combination with a light-sensitive drug, called Photofrin, to destroy diseased tissue. Patients are given an injection of the light-sensitive drug two days before their treatment. The drug is then “taken up” in the diseased tissue. On the day of treatment, and after first receiving a mild sedative, the laser light at the end of an endoscope is applied to the area. The light activates the drug and kills diseased cells without affecting normal tissue. The treatment is done as an outpatient procedure and can be repeated as needed. It takes about 45 minutes.

Photodynamic therapy is used in patients with Barrett's esophagus with low- or high-grade dysplasia, and in patients with early or advanced esophageal cancer. Patients are able to eat a nearly normal diet within four to five days of treatment. Because a light-sensitive drug is used, patients must stay out of direct sunlight for four weeks after receiving this therapy.

Radiofrequency ablation is a treatment modality commonly used in cardiology to treat uncoordinated heartbeats, called tachyarrhythmias, as well as in other medical specialties. This same radiofrequency energy—similar to microwave energy—is used in Barrett's esophagus to destroy cells within the Barrett's tissue. After the patient has received a mild sedative, the gastroenterologist gently maneuvers an endoscope down the esophagus. The endoscope contains a catheter with an electrode at its tip and a small camera that sends images to a video monitor. When the area of the esophagus has been identified, the gastroenterologist directs the radiofrequency energy at the Barrett's segment to be treated. The heat energy destroys the diseased cells, leaving healthy tissue untreated.

This minimally invasive treatment takes about 30 minutes and patients are able to resume their normal activities the following day. Some patients experience minor swallowing discomfort for several days.

### SUMMARY

In an aspect of the invention, there is an endoscopic ablation system comprising: an endoscope; an ablation optical phased array (AOPA) with the endoscope, the AOPA being configured to emit a beam in a selectively controllable direction, the beam being configured to ablate tissue; and an imaging system with the endoscope, the imaging system configured to capture images in the vicinity of the endoscope and/or the AOPA.

In an embodiment, the AOPA comprises plural antenna elements, wherein the antenna elements are optical elements.

In an embodiment, the plural antenna elements comprise between 4 and 1028 individual optical antenna elements, for example 8, 16, 32, 64, 128, 256, 512, or 1028 individual optical antenna elements.

In an embodiment, the endoscopic ablation system further comprises a control system that controls inputs to the antenna elements to perform beam forming (aka beam steering).

In an embodiment, the control system controls phases of signals input to the antenna elements to perform the beam forming (aka beam steering).

In an embodiment, the beam forming (aka beam steering) comprises forming a beam in a direction relative to a frame of reference of the plural antenna elements, the direction being defined by a polar/elevation angle and an azimuth angle relative to the frame of reference, and the control system can change the inputs to the antenna elements to change the polar/elevation angle and an azimuth angle to achieve a desired direction of the beam.

In an embodiment, the phased control of the light in the integrated waveguides is controlled via external laser phase control whose light goes through fiber optics to the endoscope head, or is controlled locally (at or near the endoscope head) using controllable optical phase shifters such as TiN heaters above integrated waveguides of liquid crystal phase shift elements above integrated waveguides.

In an embodiment, the AOPA forms a beam having a spot size of about 7 um (micrometers).

In an embodiment, the OPAs utilize 2D arrays of optical antennas, 1D arrays of optical gratings, or other permutations or optical antennas such as plasmonic based optical antennas.

In an embodiment, the AOPA is in or on the endoscope.

In an embodiment, the AOPA is in or on the endoscope and the imaging system is in or on the endoscope.

In an embodiment, the imaging system comprises one of a CCD imager, an OPA assisted imager, an OPA illuminated imager, a LiDAR imager, and an Optical coherence tomography (OCT) imager.

In an embodiment, the endoscopic ablation system further comprises an illumination system with the endoscope.

In an embodiment, the illumination system comprises an LED (light emitting diode).

In an embodiment, the illumination system comprises lighting control and/or lighting circuitry.

In an embodiment, the endoscopic ablation system further comprises a movement system for controlling movement of the endoscope.

In an embodiment, the movement system comprises at least one torque coil.

In an embodiment, the movement system comprises a steerable introducer.

In an embodiment, the endoscopic ablation system further comprises a temperature control system with the endoscope.

In an embodiment, the temperature control system comprises a heat sink.

In an embodiment, the temperature control system comprises a fluid heat sink.

In an embodiment, the endoscopic ablation system further comprises CMOS spot direction and size control chips/circuits.

In an embodiment, the endoscopic ablation system further comprises imager CCD readout circuits.

In an embodiment, the endoscopic ablation system further comprises LiDAR control circuits.

In an embodiment, the endoscopic ablation system further comprises artificial/neural network circuitry for automated control of ablation based on automated image recognition of some tissues such as colon polyps/cancers.

In an embodiment, the endoscopic ablation system is tethered via power/control lines, optical fibers, and/or fluid cooling lines to optical supply and control, electronic control, and fluid supply outside of the subject, human, animal, or area of operation.

In an embodiment, the AOPA comprises an OPA in the head of the endoscope and the imaging system comprises a separate OPA at the head of the endoscope, and the OPA and the separate OPA are formed on different chips.

In an embodiment, the AOPA comprises an OPA in the head of the endoscope and the imaging system comprises a separate OPA at the head of the endoscope, and the OPA and the separate OPA are formed on a common integrated substrate or two chips bonded to a common chip.

In an embodiment, the AOPA comprises an OPA in the head of the endoscope and the imaging system comprises a separate imager (e.g., CCD) at the head of the endoscope, and the OPA and the separate imager are formed on different chips.

In an embodiment, the endoscopic ablation system is fully self-contained, for example in a swallowable capsule that can go through the digestive track or in any other self-contained vessel within a subject, human, or animal.

In an embodiment, the endoscopic ablation system comprises a self-contained capsule, wherein the AOPA comprises an OPA on a first substrate and the imaging system comprises a separate OPA on a second substrate separate from the first substrate.

In an embodiment, the endoscopic ablation system comprises a self-contained capsule, wherein the AOPA comprises an OPA in on a first substrate and the imaging system comprises a separate OPA on the first substrate.

In an embodiment, the endoscopic ablation system comprises a self-contained capsule, wherein the AOPA comprises an OPA on a first substrate and the imaging system comprises a separate imager chip (e.g., CCD) on a second substrate separate from the first substrate.

In an embodiment, the capsule comprises one or more of: batteries, capacitors, imaging and ablation control electronics, communication electronics, AI circuitry, laser generation electronics, and optics.

In an embodiment, a method comprises using any of the aforementioned embodiments of the endoscopic ablation system for ablation of living (human/animal) tissue using the AOPA.

In an embodiment, a method comprises using any of the aforementioned embodiments of the endoscopic ablation system for microsurgery in living tissues by taking advantage of the small optical ablation spot possible with the AOPA.

In an embodiment, a method comprises using any of the aforementioned embodiments of the endoscopic ablation system including utilizing the imaging system (e.g., LIDAR, CCD imager chip, sonogram, etc.) in conjunction with the AOPA for real-time feedback during microsurgery.

In an embodiment, a method comprises using any of the aforementioned embodiments of the endoscopic ablation system including using the optical phased array tissue ablation over large areas of tissue in a programmed pattern: for example, for resurfacing of tissue over a larger area as opposed to concentrating on the ablation on a single small, high-power spot and moving the beam.

In an embodiment, a method comprises using any of the aforementioned embodiments of the endoscopic ablation system including using an optical phased array tissue ablation system to move adjust the location, spot size, optical power, etc. such detailed surgical procedures can be achieved.

In an embodiment, a method comprises using any of the aforementioned embodiments of the endoscopic ablation system including using automated/semi-automated systems for calculating surgical recipes for the optical phased array tissue ablation sequence given an image of the target tissue with input from surgeon describing desired pattern (using inputs such as a mouse or touchpad, etc.).

In an embodiment, the endoscopic ablation system includes an automated feedback system between the imaging system and the AOPA to control ablation to desired target depths, power, and spot size.

### BRIEF DESCRIPTION OF THE DRAWINGS

The present invention is described in the detailed description which follows, in reference to the noted plurality of drawings by way of non-limiting examples of exemplary embodiments of the present invention.

FIG. 1 shows an endoscopic ablation system in accordance with aspects of the present invention.

FIG. 2 shows an endoscopic ablation system in accordance with aspects of the present invention.

FIG. 3 shows an endoscopic ablation system in accordance with aspects of the present invention.

FIG. 4 shows an endoscopic ablation system in accordance with aspects of the present invention.

FIG. 5 shows an endoscopic ablation system in accordance with aspects of the present invention.

FIG. 6 shows an endoscopic ablation system in accordance with aspects of the present invention.

FIG. 7 shows an optical phased array in accordance with aspects of the present invention.

FIG. 8A illustrates an exemplary method of tissue ablation in accordance with aspects of the present invention.

FIG. 8B illustrates an exemplary method of tissue ablation in accordance with aspects of the present invention.

FIG. 8C illustrates an exemplary method of tissue ablation in accordance with aspects of the present invention.

FIG. 8D illustrates an exemplary method of tissue ablation in accordance with aspects of the present invention.

### DETAILED DESCRIPTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

The present invention relates generally to medical devices and methods of use and, more particularly, to devices and methods for endoscopic coherent tissue ablation. Implementations of the present disclosure provide a novel endoscopic design which uses coherent optical power to ablate tissue which can be used in humans or animals: Coherent Optical Tissue Ablation (COTA).

Systems in accordance with aspects of the present invention are capable of ablating tissue in the human body with incredible accuracy. Integrated optical-phased arrays (OPAs) can control beam sizes in the near field to less than 7 um spot sizes, making it advantageous for procedures requiring high precision such as some eye surgeries and other tissue ablation.

An endoscope fitted with this technology in accordance with aspects of the present invention can perform the surgery/ablation in real time under the direction of a surgeon or under the direction of an artificial intelligence (AI) imaging/surgical assist algorithm.

Optical-phased arrays (OPAs) have been contemplated and demonstrated for use with endoscopes in terms of their imaging capabilities, for example using them similar to how LiDAR is used in autonomous vehicles or for aerial imaging. OPAs present unique capabilities in that they can be steered with no moving parts, and because they can image using coherent light, their possible imaging techniques are broader than those using only incoherent light.

Implementations of the present disclosure use coherent optical tissue ablation (COTA) to remove tissue using an endoscopic device. This has advantages over RF ablation used in terms of precision control and can be used alongside an OPA or CCD imager.

Systems in accordance with aspects of the present invention also have the advantage of being able to steer the ablation spot location on the target tissue within the field of vision of the endoscopic imager (whether using CCD imagers or OPA/Lidar-type imaging) without moving the endoscope head (or self-contained capsule) and requiring no moving lens/parts.

There are various ways of fabricating the OPAs that could be used in the COTA systems in accordance with aspects of the present invention. They could be highly integrated consisting of single substrates or multiple integrated substrates with CMOS control. The controllability of the beam direction allows, ideally, a large range of azimuthal and elevation angular control of the spot direction relative to the endoscopic head direction. However, even with somewhat restricted directionality (for example, even if only one angular direction is controllable, as in some linear optical antenna arrays), the system would be very useful. The OPAs used can consist of a wide range of optical antenna elements (e.g., 512, 1028, 10000, etc.). In embodiments, the phased control of the light in the integrated waveguides is steered via external laser phase control whose light goes through fiber optics to the endoscope head, or is controlled locally (at or near the endoscope head) using controllable optical phase shifters such as TiN heaters above integrated waveguides of liquid crystal phase shift elements above integrated waveguides. In embodiments, these OPAs utilize 2D arrays of optical antennas 1D arrays of optical gratings or other permutations or optical antennas such as plasmonic based optical antennas, etc.

A benefit of systems in accordance with aspects of the present invention is the beam/spot directionally control range and precision compared to other existing endoscopic surgical/ablation heads, which makes it ideal for the most delicate micro-surgeries.

In embodiments, the endoscope heads in systems in accordance with aspects of the present invention comprise/house many other system control elements which may include, but are not limited to, temperature control (e.g., via fluid heat sinks, etc.), CMOS spot direction and size control chips/circuits, imager CCD readout circuits, lighting control and lighting circuitry, for example, using LED lighting, LiDAR control circuits, and artificial/neural network circuitry for automated control of ablation based on automated image recognition of some tissues such as colon polyps/cancers.

In embodiments, the system is fully self contained, for example in a swallowable capsule that can go through the digestive track or in any other self-contained vessel within a subject, human, or animal. In embodiments, the system is tethered via power/control lines, optical fibers, and/or fluid cooling lines to optical supply and control, electronic control, and fluid supply outside of the subject, human, animal, or area of operation.

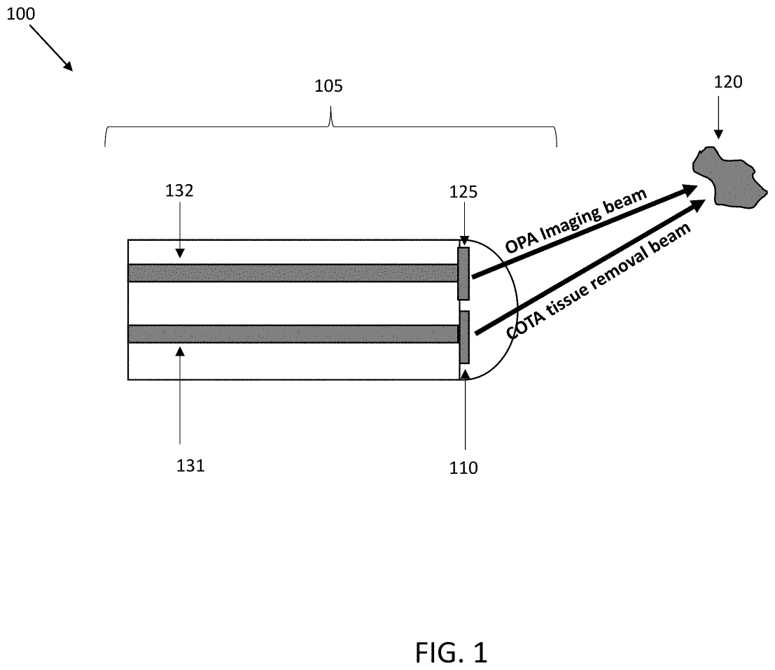

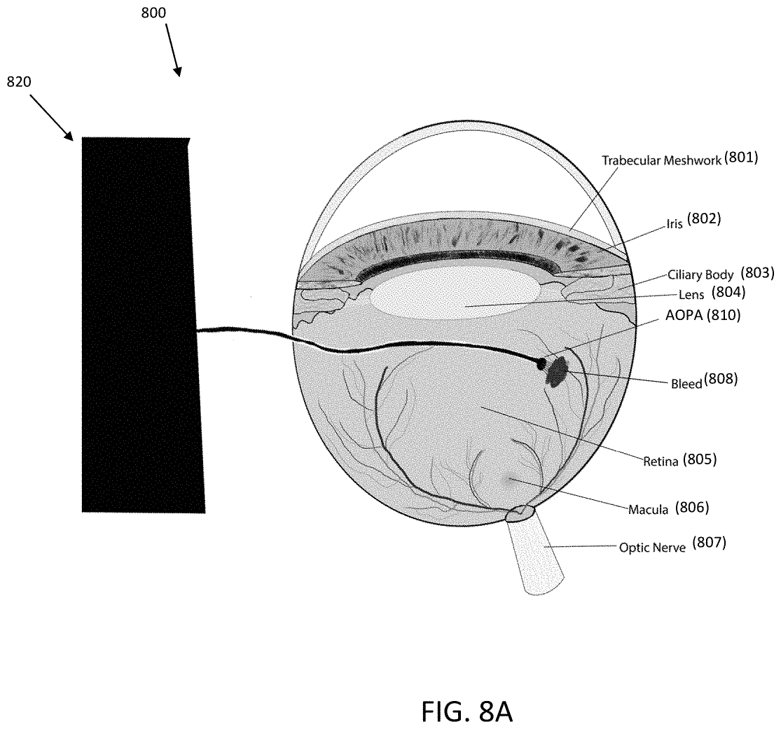

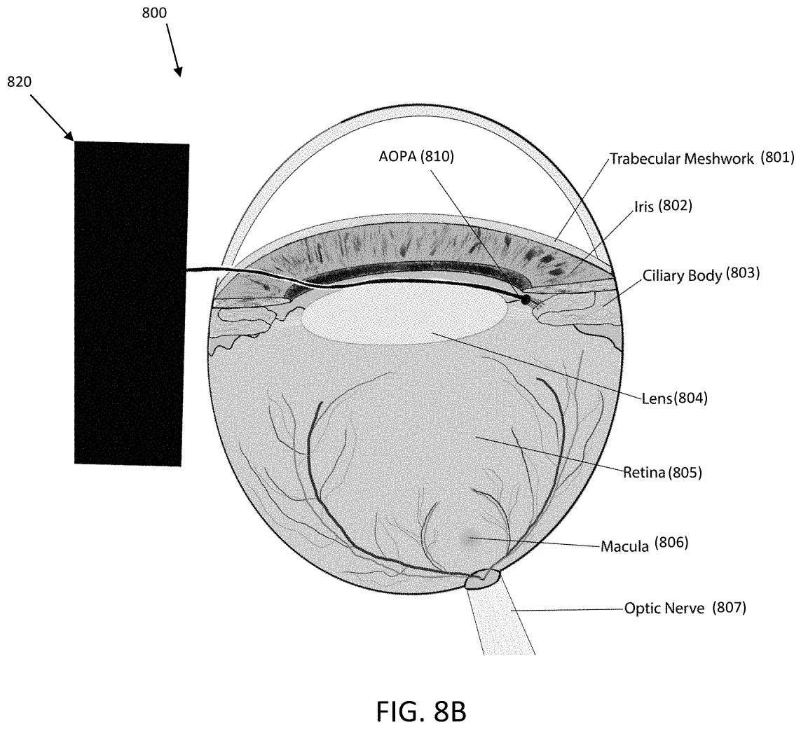

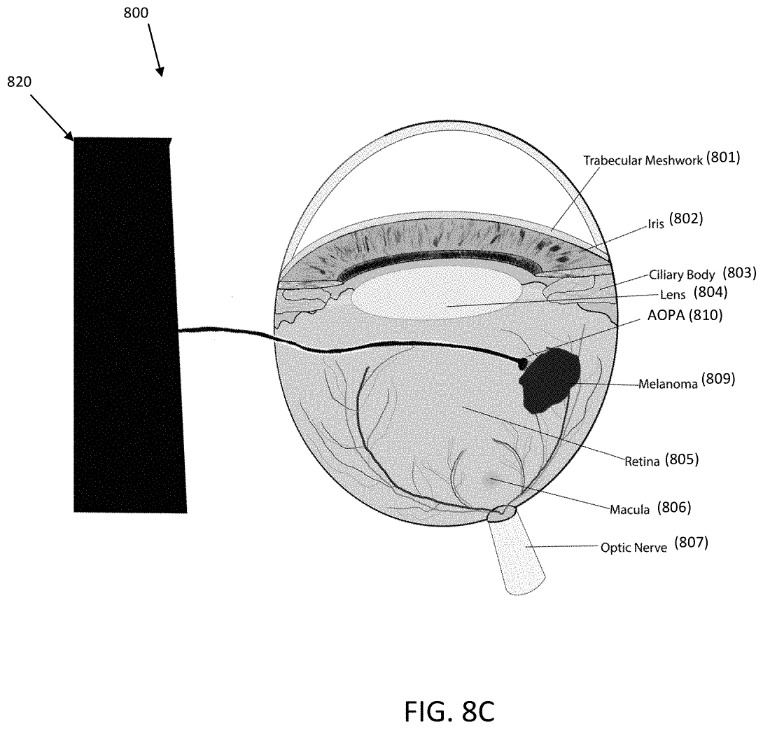

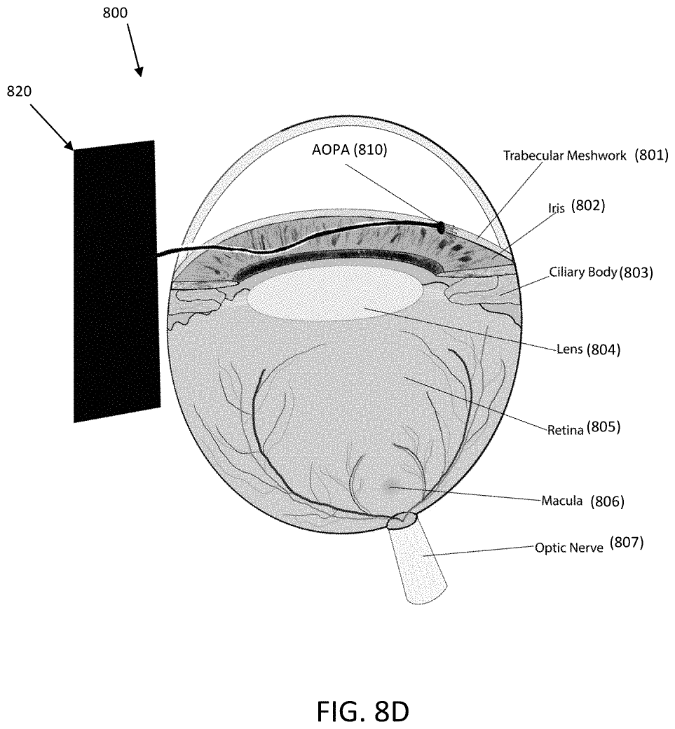

FIGS. 1-6, 7, and 8A-D illustrate aspects of the disclosure. FIGS. 1-6 illustrate embodiments of COTA systems in accordance with aspects of the invention. FIGS. 8A-D illustrate exemplary methods in accordance with aspects of the invention. The methods of FIGS. 8A-D may use any of the COTA systems of FIGS. 1-6.

FIG. 1 shows an endoscopic ablation system 100 in accordance with aspects of the present invention. In particular, FIG. 1 shows an endoscope head (or tip) 105 that includes an ablation optical phased array (AOPA) 110 configured to emit a beam in a selectively controllable direction, the beam being configured to ablate a tissue 120 which may be a target tissue inside the body of a human or other animal. In embodiments, the endoscope head 105 is connected to (e.g., part of) an endoscope that can be manipulated by a user to look inside a body or a human or other animal.

In embodiments, the endoscope head 105 includes an imaging system configured to capture images in the vicinity of the endoscope head 105 and/or the AOPA 110. In the embodiment shown in FIG. 1, the imaging system comprises an imager chip 125 that includes a second optical phased array that is configured to capture real-time images of the tissue 120 using optical phased array imaging techniques. In embodiments, the second optical phased array is separate from the AOPA 110. In embodiments, the imager chip 125 and a chip containing the AOPA 110 are different chips.

In embodiments, the beam emitted by the AOPA 110 comprises a beam of light. In embodiments, the real-time images captured by the imaging system (e.g., imager chip 125) provide real-time feedback to a user controlling the AOPA 110 by showing where the beam is impacting the body relative to the tissue 120. In this manner, based on the real-time images provided by the imaging system, the user may provide control inputs to control the direction of the beam emitted by the AOPA 110 so that the beam is directed to its intended target, e.g., the tissue 120.

In embodiments, the endoscope head 105 includes first connection 131 that is operatively connected to the AOPA 110 for providing control signals to the AOPA 110 for controlling the direction of the bean emitted by the AOPA 110. The first connection 131 may comprise fiber optic and/or electronic signal paths, and may extend from the endoscope head 105 through an endoscope body (not shown) and to a device, such as a computing device, that is configured to receive user input for changing the direction of the beam emitted by the AOPA 110.

In embodiments, the endoscope head 105 includes a second connection 132 that is operatively connected to the imager chip 125 for providing control signals to and receiving image signals from the imager chip 125. The second connection 132 may comprise fiber optic and/or electronic signal paths, and may extend from the endoscope head 105 through an endoscope body (not shown) and to a device that is configured to receiving image signals from the imager chip 125, such as a computing device that displays the images on a display that is visible to a user operating the endoscope.

In embodiments, the endoscopic ablation system 100 further comprises an illumination system with the endoscope, e.g., at the endoscope head 105. In an embodiment, the illumination system comprises an LED (light emitting diode). In an embodiment, the illumination system comprises lighting control and/or lighting circuitry. In embodiments, the illumination system is configured to illuminate an area that is within the field of view of the imaging system.

In an embodiment, the endoscopic ablation system 100 further comprises a movement system for controlling movement of the endoscope. In an embodiment, the movement system comprises at least one torque coil. In an embodiment, the movement system comprises a steerable introducer. In embodiments, the movement system is configured to control movement of the endoscope (e.g., maneuverability, pushability, etc.) so that a user can guide the endoscope inside the body of the person or animal that is undergoing tissue ablation therapy.

In an embodiment, the endoscopic ablation system 100 further comprises a temperature control system with the endoscope. In an embodiment, the temperature control system comprises a heat sink. In an embodiment, the temperature control system comprises a fluid heat sink.

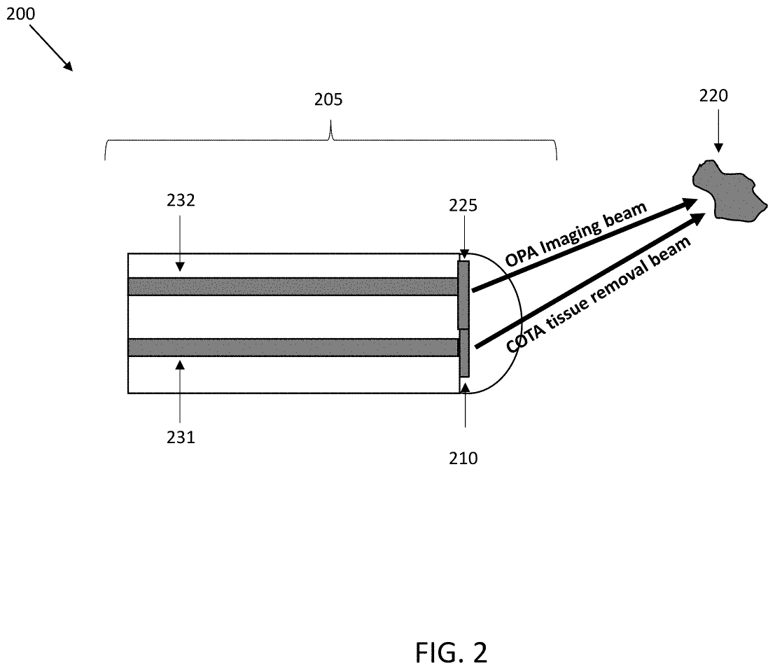

FIG. 2 shows an endoscopic ablation system 200 in accordance with aspects of the present invention. In particular, FIG. 2 shows an endoscope head (or tip) 205 that includes an ablation optical phased array (AOPA) 210 configured to emit a beam in a selectively controllable direction, the beam being configured to ablate a tissue 220 which may be a target tissue inside the body of a human or other animal. In embodiments, the endoscope head 205 is connected to (e.g., part of) an endoscope that can be manipulated by a user to look inside a body or a human or other animal.

In embodiments, the endoscope head 205 includes an imaging system configured to capture images in the vicinity of the endoscope head 205 and/or the AOPA 210. In the embodiment shown in FIG. 2, the imaging system comprises an imager chip 225 that includes a second optical phased array that is configured to capture real-time images of the tissue 220 using optical phased array imaging techniques. In embodiments, the second optical phased array is separate from the AOPA 210. In embodiments, the imager chip 225 and a chip containing the AOPA 210 are the same chip (e.g., the imaging system and the AOPA 210 are formed on a common integrated substrate) or are two chips bonded to a common chip.

In embodiments, the beam emitted by the AOPA 210 comprises a beam of light. In embodiments, the real-time images captured by the imaging system (e.g., imager chip 225) provide real-time feedback to a user controlling the AOPA 210 by showing where the beam is impacting the body relative to the tissue 220. In this manner, based on the real-time images provided by the imaging system, the user may provide control inputs to control the direction of the beam emitted by the AOPA 210 so that the beam is directed to its intended target, e.g., the tissue 220.

In embodiments, the endoscope head 205 includes first connection 231 that is operatively connected to the AOPA 210 for providing control signals to the AOPA 210 for controlling the direction of the bean emitted by the AOPA 210. The first connection 231 may comprise fiber optic and/or electronic signal paths, and may extend from the endoscope head 205 through an endoscope body (not shown) and to a device that is configured to receive user input for changing the direction of the beam emitted by the AOPA 210, such as a computing device.

In embodiments, the endoscope head 205 includes a second connection 232 that is operatively connected to the imager chip 225 for providing control signals to and receiving image signals from the imager chip 225. The second connection 232 may comprise fiber optic and/or electronic signal paths, and may extend from the endoscope head 205 through an endoscope body (not shown) and to a device that is configured to receiving image signals from the imager chip 225, such as a computing device that displays the images on a display that is visible to a user operating the endoscope.

In embodiments, the endoscopic ablation system 200 further comprises an illumination system with the endoscope, e.g., at the endoscope head 205. In an embodiment, the illumination system comprises an LED (light emitting diode). In an embodiment, the illumination system comprises lighting control and/or lighting circuitry. In embodiments, the illumination system is configured to illuminate an area that is within the field of view of the imaging system.

In an embodiment, the endoscopic ablation system 200 further comprises a movement system for controlling movement of the endoscope. In an embodiment, the movement system comprises at least one torque coil. In an embodiment, the movement system comprises a steerable introducer. In embodiments, the movement system is configured to control movement of the endoscope (e.g., maneuverability, pushability, etc.) so that a user can guide the endoscope inside the body of the person or animal that is undergoing tissue ablation therapy.

In an embodiment, the endoscopic ablation system 200 further comprises a temperature control system with the endoscope. In an embodiment, the temperature control system comprises a heat sink. In an embodiment, the temperature control system comprises a fluid heat sink.

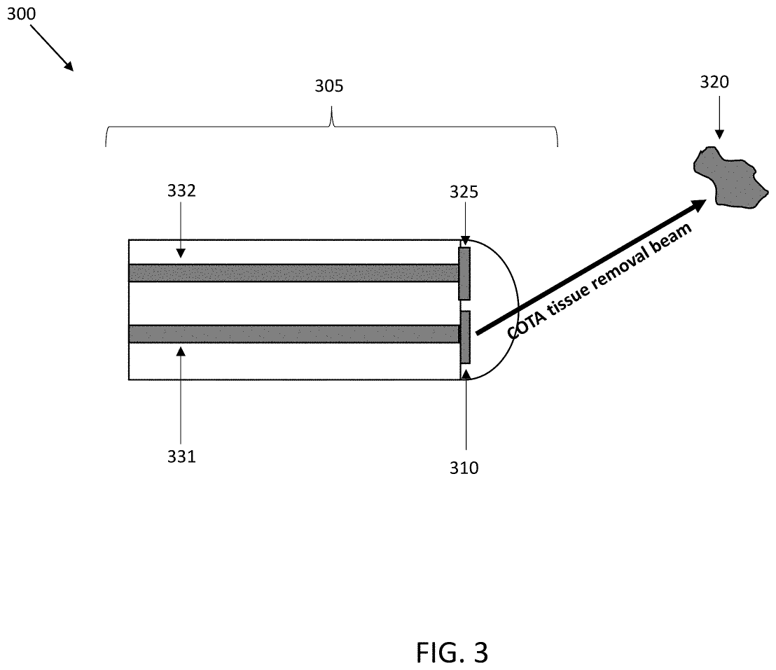

FIG. 3 shows an endoscopic ablation system 300 in accordance with aspects of the present invention. In particular, FIG. 3 shows an endoscope head (or tip) 305 that includes an ablation optical phased array (AOPA) 310 configured to emit a beam in a selectively controllable direction, the beam being configured to ablate a tissue 320 which may be a target tissue inside the body of a human or other animal. In embodiments, the endoscope head 305 is connected to (e.g., part of) an endoscope that can be manipulated by a user to look inside a body or a human or other animal.

In embodiments, the endoscope head 305 includes an imaging system configured to capture images in the vicinity of the endoscope head 305 and/or the AOPA 310. In the embodiment shown in FIG. 3, the imaging system comprises an imager chip 325 that includes a LIDAR, CCD imager chip, or sonogram, for example, that is configured to capture real-time images of the tissue 320. In embodiments, the imaging system is separate from the AOPA 310. In embodiments, the imager chip 325 and a chip containing the AOPA 310 are different chips.

In embodiments, the beam emitted by the AOPA 310 comprises a beam of light. In embodiments, the real-time images captured by the imaging system (e.g., imager chip 325) provide real-time feedback to a user controlling the AOPA 310 by showing where the beam is impacting the body relative to the tissue 320. In this manner, based on the real-time images provided by the imaging system, the user may provide control inputs to control the direction of the beam emitted by the AOPA 310 so that the beam is directed to its intended target, e.g., the tissue 320.

In embodiments, the endoscope head 305 includes first connection 331 that is operatively connected to the AOPA 310 for providing control signals to the AOPA 310 for controlling the direction of the bean emitted by the AOPA 310. The first connection 331 may comprise fiber optic and/or electronic signal paths, and may extend from the endoscope head 305 through an endoscope body (not shown) and to a device that is configured to receive user input for changing the direction of the beam emitted by the AOPA 310, such as a computing device.

In embodiments, the endoscope head 305 includes a second connection 332 that is operatively connected to the imager chip 325 for providing control signals to and receiving image signals from the imager chip 325. The second connection 332 may comprise fiber optic and/or electronic signal paths, and may extend from the endoscope head 305 through an endoscope body (not shown) and to a device that is configured to receiving image signals from the imager chip 325, such as a computing device that displays the images on a display that is visible to a user operating the endoscope.

In embodiments, the endoscopic ablation system 300 further comprises an illumination system with the endoscope, e.g., at the endoscope head 305. In an embodiment, the illumination system comprises an LED (light emitting diode). In an embodiment, the illumination system comprises lighting control and/or lighting circuitry. In embodiments, the illumination system is configured to illuminate an area that is within the field of view of the imaging system.

In an embodiment, the endoscopic ablation system 300 further comprises a movement system for controlling movement of the endoscope. In an embodiment, the movement system comprises at least one torque coil. In an embodiment, the movement system comprises a steerable introducer. In embodiments, the movement system is configured to control movement of the endoscope (e.g., maneuverability, pushability, etc.) so that a user can guide the endoscope inside the body of the person or animal that is undergoing tissue ablation therapy.

In an embodiment, the endoscopic ablation system 300 further comprises a temperature control system with the endoscope. In an embodiment, the temperature control system comprises a heat sink. In an embodiment, the temperature control system comprises a fluid heat sink.

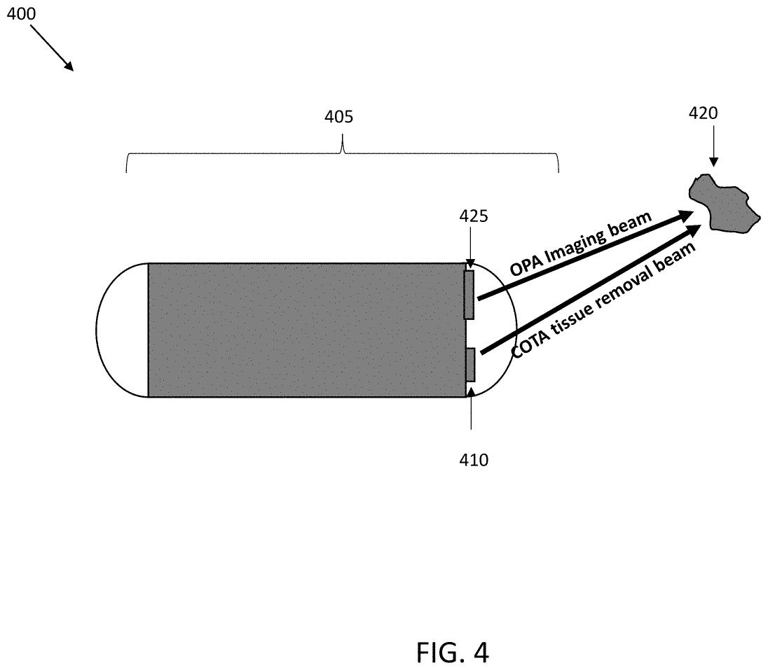

FIG. 4 shows an endoscopic ablation system 400 in accordance with aspects of the present invention. In particular, FIG. 4 shows a capsule 405 that is swallowable by a human or other animal and configured to go through the digestive track of the human or other animal. For example, the capsule 405 may be about the size of a large vitamin pill, e.g., about 1 inch in length.

In embodiments, the capsule 405 includes an ablation optical phased array (AOPA) 410 configured to emit a beam in a selectively controllable direction, the beam being configured to ablate a tissue 420 which may be a target tissue inside the body of a human or other animal.

In embodiments, the capsule 405 includes an imaging system configured to capture images in the vicinity of the capsule 405 and/or the AOPA 410. In the embodiment shown in FIG. 4, the imaging system comprises an imager chip 425 that includes a second optical phased array that is configured to capture real-time images of the tissue 420 using optical phased array imaging techniques. In embodiments, the second optical phased array is separate from the AOPA 410. In embodiments, the imager chip 425 and a chip containing the AOPA 410 are different chips. The capsule 405 may include plural pairs of AOPA 410 and imager chip 425, for example one pair at each end of the capsule 405.

In embodiments, the beam emitted by the AOPA 410 comprises a beam of light. In embodiments, the real-time images captured by the imaging system (e.g., imager chip 425) provide real-time feedback to a user controlling the AOPA 410 by showing where the beam is impacting the body relative to the tissue 420. In this manner, based on the real-time images provided by the imaging system, the user may provide control inputs to control the direction of the beam emitted by the AOPA 410 so that the beam is directed to its intended target, e.g., the tissue 420.

In embodiments, the capsule 405 comprises one or more of: batteries, capacitors, imaging and ablation control electronics, communication electronics, AI circuitry, laser generation electronics, and optics. The communication electronics may provide wireless communication between the capsule 405 and an external device (e.g., computing system) outside the body of the human or other animal that swallowed the capsule 405. The external device may be configured to display real-time images captured by the imaging system. The external device may be configured to receive user input, and to transmit signals to the capsule 405 based on this input, to control the direction of the beam emitted by the AOPA 410 so that the beam is directed to its intended target, e.g., the tissue 420.

In embodiments, the endoscopic ablation system 400 further comprises an illumination system with the capsule 405. In an embodiment, the illumination system comprises an LED (light emitting diode). In an embodiment, the illumination system comprises lighting control and/or lighting circuitry. In embodiments, the illumination system is configured to illuminate an area that is within the field of view of the imaging system.

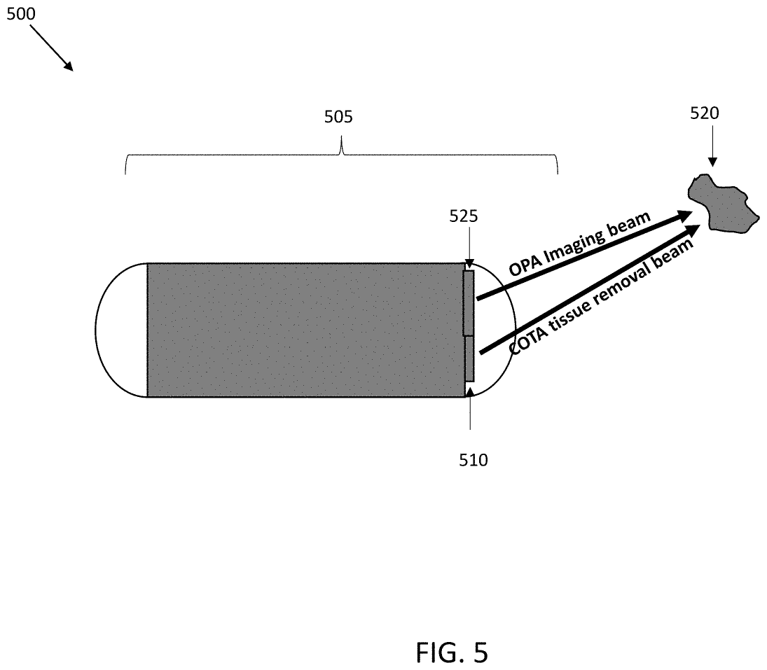

FIG. 5 shows an endoscopic ablation system 500 in accordance with aspects of the present invention. In particular, FIG. 5 shows a capsule 505 that is swallowable by a human or other animal and configured to go through the digestive track of the human or other animal. For example, the capsule 505 may be about the size of a large vitamin pill, e.g., about 1 inch in length.

In embodiments, the capsule 505 includes an ablation optical phased array (AOPA) 510 configured to emit a beam in a selectively controllable direction, the beam being configured to ablate a tissue 520 which may be a target tissue inside the body of a human or other animal.

In embodiments, the capsule 505 includes an imaging system configured to capture images in the vicinity of the capsule 505 and/or the AOPA 510. In the embodiment shown in FIG. 5, the imaging system comprises an imager chip 525 that includes a second optical phased array that is configured to capture real-time images of the tissue 520 using optical phased array imaging techniques. In embodiments, the second optical phased array is separate from the AOPA 510. In embodiments, the imager chip 525 and a chip containing the AOPA 510 are the same chip (e.g., the imaging system and the AOPA 510 are formed on a common integrated substrate) or are two chips bonded to a common chip. The capsule 505 may include plural pairs of AOPA 510 and imager chip 525, for example one pair at each end of the capsule 505.

In embodiments, the beam emitted by the AOPA 510 comprises a beam of light. In embodiments, the real-time images captured by the imaging system (e.g., imager chip 525) provide real-time feedback to a user controlling the AOPA 510 by showing where the beam is impacting the body relative to the tissue 520. In this manner, based on the real-time images provided by the imaging system, the user may provide control inputs to control the direction of the beam emitted by the AOPA 510 so that the beam is directed to its intended target, e.g., the tissue 520.

In embodiments, the capsule 505 comprises one or more of: batteries, capacitors, imaging and ablation control electronics, communication electronics, AI circuitry, laser generation electronics, and optics. The communication electronics may provide wireless communication between the capsule 505 and an external device (e.g., computing system) outside the body of the human or other animal that swallowed the capsule 505. The external device may be configured to display real-time images captured by the imaging system. The external device may be configured to receive user input, and to transmit signals to the capsule 505 based on this input, to control the direction of the beam emitted by the AOPA 510 so that the beam is directed to its intended target, e.g., the tissue 520.

In embodiments, the endoscopic ablation system 500 further comprises an illumination system with the capsule 505. In an embodiment, the illumination system comprises an LED (light emitting diode). In an embodiment, the illumination system comprises lighting control and/or lighting circuitry. In embodiments, the illumination system is configured to illuminate an area that is within the field of view of the imaging system.

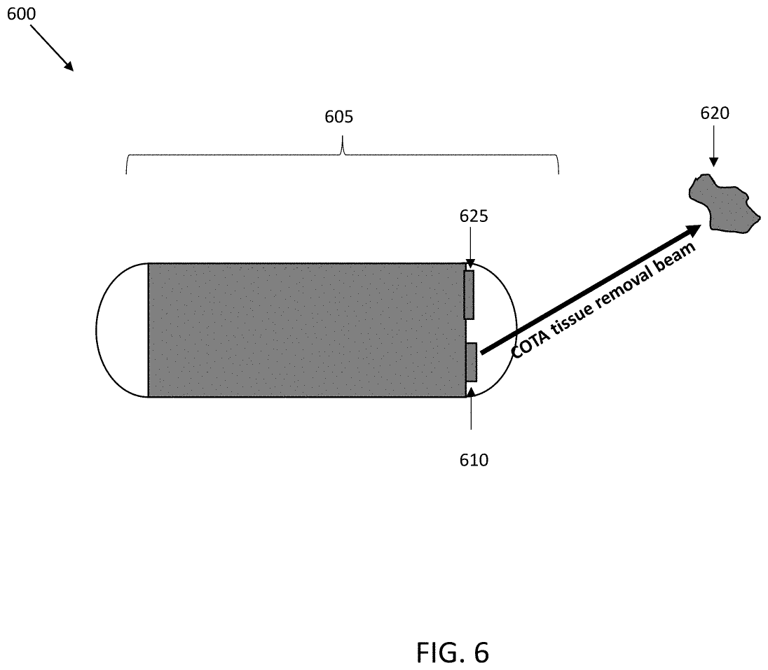

FIG. 6 shows an endoscopic ablation system 600 in accordance with aspects of the present invention. In particular, FIG. 6 shows a capsule 605 that is swallowable by a human or other animal and configured to go through the digestive track of the human or other animal. For example, the capsule 605 may be about the size of a large vitamin pill, e.g., about 1 inch in length.

In embodiments, the capsule 605 includes an ablation optical phased array (AOPA) 610 configured to emit a beam in a selectively controllable direction, the beam being configured to ablate a tissue 620 which may be a target tissue inside the body of a human or other animal.

In embodiments, the capsule 605 includes an imaging system configured to capture images in the vicinity of the capsule 605 and/or the AOPA 610. In the embodiment shown in FIG. 6, the imaging system comprises an imager chip 625 that includes a LIDAR, CCD imager chip, or sonogram, for example, that is configured to capture real-time images of the tissue 620. In embodiments, the imaging system is separate from the AOPA 610. In embodiments, the imager chip 625 and a chip containing the AOPA 610 are different chips. The capsule 605 may include plural pairs of AOPA 610 and imager chip 625, for example one pair at each end of the capsule 605.

In embodiments, the beam emitted by the AOPA 610 comprises a beam of light. In embodiments, the real-time images captured by the imaging system (e.g., imager chip 625) provide real-time feedback to a user controlling the AOPA 610 by showing where the beam is impacting the body relative to the tissue 620. In this manner, based on the real-time images provided by the imaging system, the user may provide control inputs to control the direction of the beam emitted by the AOPA 610 so that the beam is directed to its intended target, e.g., the tissue 620.

In embodiments, the capsule 605 comprises one or more of: batteries, capacitors, imaging and ablation control electronics, communication electronics, AI circuitry, laser generation electronics, and optics. The communication electronics may provide wireless communication between the capsule 605 and an external device (e.g., computing system) outside the body of the human or other animal that swallowed the capsule 605. The external device may be configured to display real-time images captured by the imaging system. The external device may be configured to receive user input, and to transmit signals to the capsule 605 based on this input, to control the direction of the beam emitted by the AOPA 610 so that the beam is directed to its intended target, e.g., the tissue 620.

In embodiments, the endoscopic ablation system 600 further comprises an illumination system with the capsule 605. In an embodiment, the illumination system comprises an LED (light emitting diode). In an embodiment, the illumination system comprises lighting control and/or lighting circuitry. In embodiments, the illumination system is configured to illuminate an area that is within the field of view of the imaging system.

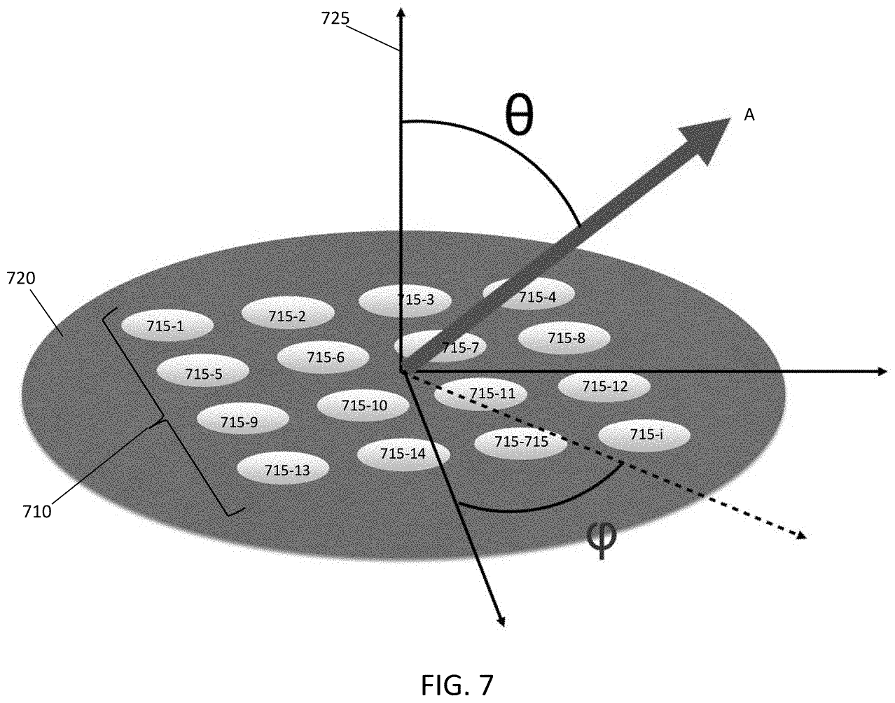

FIG. 7 shows an ablation optical phased array (AOPA) in accordance with aspects of the present invention. In the example shown in FIG. 7, the AOPA 710 comprises a 4×4 array of antenna elements 715-1, 715-2, . . . , 715-i included in a sensor 720. In this example “i” equals sixteen; however, the number of antenna elements shown in FIG. 7 is not intended to be limiting, and the AOPA 710 may have a different number of antenna elements. Similarly, the implementation in the sensor 720 is only for illustrative purposes, and the AOPA 710 may be implemented in different structures.

Still referring to FIG. 7, the arrow A represents a direction of the beam that is formed by the AOPA 710 using constructive and destructive superposition of signals from the antenna elements 715-1, 715-2, . . . , 715-i using beam steering principles. Angle θ represents the polar angle and angle φ represents the azimuth angle of the direction of the arrow A relative to a frame of reference 725 defined with respect to the AOPA 710.

In this manner, an endoscope ablation system in accordance with aspects of the invention may include an ablation optical phased array (AOPA) with the endoscope, the AOPA being configured to emit a beam of light in a selectively controllable direction, wherein the direction is selectively controlled using beam forming (aka beam steering) comprising: forming a beam in a direction (A) relative to a frame of reference (725) of the plural antenna elements 715-1, 715-2, . . . , 715-i, the direction (A) being defined by a polar/elevation angle (θ) and an azimuth angle (φ) relative to the frame of reference. In embodiments a control system changes the inputs to the antenna elements 715-1, 715-2, . . . , 715-i to change the polar/elevation angle (θ) and an azimuth angle (φ) to achieve the desired direction (A) of the beam to perform beam forming (aka beam steering). In embodiments, the control system controls phases of signals input to the antenna elements 715-1, 715-2, . . . , 715-i to perform the beam forming (aka beam steering). In this manner, the AOPAs of FIGS. 1-6 (e.g., AOPA 110, 210, 310, 410, 510, 610) can change the direction of the beam used for tissue ablation without moving any parts in the endoscope.

FIGS. 8A-D illustrate exemplary methods of tissue ablation in accordance with aspects of the present invention. FIGS. 8A-D show an eye including a trabecular meshwork 801, iris 802, ciliary body 803, lens 804, retina 805, macula 806, and optic nerve 807. FIGS. 8A-D also show an endoscopic ablation system 800 including an AOPA 810. The endoscopic ablation system 800 may comprise one of the endoscopic ablation systems 100, 200, or 300, and the AOPA 810 may comprise one of the AOPAs 110, 210, 310. The endoscopic ablation system 800 may comprise an external device 820 external to the eye. The external device 820 may include a display that displays real time images captured by the imaging system of the endoscopic ablation system 800. The external device 820 may be configured to receive user input to control the direction of the beam emitted by the AOPA 810. In this manner, the endoscopic ablation system 800 including the AOPA 810 can be used to direct a precise, powerful beam of energy directly to targeted areas in the eye.

In the example shown in FIG. 8A, the eye has a bleed 808 in the retina 805. In this example, the AOPA 810 is used to direct a precise, powerful beam of energy directly to bleeding 808 in the retina 805. Precise ablation of actively bleeding vessels and/or lesions can preserve surrounding tissue and provide better visual outcomes. This can be done to any lesion in the retina that is at risk of rupture. Prophylactic ablation can also be performed on retinal tears and other areas of weakness to prevent extension of those tears leading to retinal detachment. In embodiments, a method of tissue ablation shown in FIG. 8A includes:
1.) A small incision (sclerotomy) is made at pars plana.
2.) The AOPA 810 of the system 800 is inserted through the small incision and directed to the site of bleeding 808.
3.) Energy is applied via the AOPA 810 to completely cauterize the source of bleeding 808.

FIG. 8B illustrates an exemplary method of tissue ablation in accordance with aspects of the present invention. In this example, the AOPA 810 is used to direct a precise, powerful beam of energy directly to the ciliary body in the eye to reduce aqueous production and help to decrease intraocular pressure thereby effectively reducing damage caused by glaucoma. Being able to deliver small amounts of energy allows for titrating the amount of tissue ablated. Preserving ciliary body tissue prevents hypotony and at the same time allows enough aqueous to be produced to supply other structures with nutrients and maintaining physiologically normal eye pressure. In embodiments, a method of tissue ablation shown in FIG. 8B includes:
1.) A small incision is made at the limbus of the eye.
2.) The AOPA 810 of the system 800 is inserted through the small incision and directed between the iris 802 and the lens 804.
3.) Once the ciliary body 803 is identified, a precise amount of energy is applied to the ciliary body 803, using the AOPA 810 of the system 800, in order to ablate enough tissue to reduce aqueous production.
4.) The process can be repeated for 180 degrees or more if needed.

Selective ablation with the AOPA 810 of the system 800 leaves the eye healthy enough to allow several treatments if needed. If the intraocular pressure is still elevated after the healing period, the patient can come back and ablate more until a desired pressure is achieved.

FIG. 8C illustrates an exemplary method of tissue ablation in accordance with aspects of the present invention. In this example, the AOPA 810 is used to direct a precise, powerful beam of energy directly to melanoma 809 in the retina 805. Precise delivery of energy can directly destroy melanomas originating from the retina and choroid thereby preserving the eye and visual function. This can be applied to other malignancies of the eye. In embodiments, a method of tissue ablation shown in FIG. 8C includes:
1.) A small incision (sclerotomy) is made at pars plana.
2.) The AOPA 810 of the system 800 is inserted through the small incision and directed to the melanoma 809.
3.) Energy is applied via the AOPA 810 to completely ablate the melanoma 809 and preserve surrounding healthy tissue.

FIG. 8D illustrates an exemplary method of tissue ablation in accordance with aspects of the present invention. In this example, the AOPA 810 is used to direct a precise, powerful beam of energy directly to the trabecular meshwork 801 in the eye to increase aqueous drainage and help to decrease intraocular pressure thereby effectively reducing damage caused by glaucoma. In embodiments, a method of tissue ablation shown in FIG. 8C includes:
1.) A small incision is made at the limbus of the eye.
2.) The AOPA 810 of the system 800 is inserted through the small incision and directed on top of the iris 802 to the angle.
3.) Once the trabecular meshwork 801 is identified, a precise amount of energy is applied via the AOPA 810 to the trabecular meshwork 801 to create drainage channels to allow more aqueous into Schlemm's canal and further into collector channels lower intraocular pressure.
4.) The process can be repeated for 180 degrees or more if needed.

Selective ablation with the AOPA 810 of the system 800 leaves the eye healthy enough to allow several treatments if needed. If the intraocular pressure is still elevated after the healing period, the patient can come back and ablate more until a desired pressure is achieved.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. An endoscopic ablation system comprising:
an endoscope;
an ablation optical phased array (AOPA) with the endoscope, the AOPA being configured to emit a beam in a selectively controllable direction, the beam being configured to ablate tissue; and
an imaging system with the endoscope, the imaging system configured to capture images in the vicinity of the endoscope and/or the AOPA.

2. The endoscopic ablation system of claim 1, wherein the AOPA comprises plural antenna elements.

3. The endoscopic ablation system of claim 2, further comprising a control system that controls inputs to the antenna elements to perform beam steering.

4. The endoscopic ablation system of claim 3, wherein the control system controls phases of signals input to the antenna elements to perform the beam steering.

5. The endoscopic ablation system of claim 1, wherein the AOPA forms the beam having a spot size of about 7 micrometers.

6. The endoscopic ablation system of claim 1, wherein the AOPA utilizes 2D arrays of optical antennas, 1D arrays of optical gratings, or plasmonic based optical antennas.

7. The endoscopic ablation system of claim 1, wherein the AOPA is in or on the endoscope.

8. The endoscopic ablation system of claim 1, wherein the AOPA is in or on the endoscope and the imaging system is in or on the endoscope.

9. The endoscopic ablation system of claim 1, wherein the imaging system comprises one of a CCD imager, an OPA assisted imager, an OPA illuminated imager, a LiDAR imager, and an Optical coherence tomography (OCT) imager.

10. The endoscopic ablation system of claim 1, further comprising an illumination system with the endoscope.

11. The endoscopic ablation system of claim 1, further comprising a movement system for controlling movement of the endoscope.

12. The endoscopic ablation system of claim 1, further comprising a temperature control system with the endoscope.

13. The endoscopic ablation system of claim 1, further comprising artificial/neural network circuitry for automated control of ablation based on automated image recognition of some tissues.

14. The endoscopic ablation system of claim 1, wherein the system is tethered via power/control lines, optical fibers, and/or fluid cooling lines to optical supply and control, electronic control, and fluid supply outside of the subject, human, animal, or area of operation.

15. The endoscopic ablation system of claim 1, wherein the AOPA comprises an OPA in the head of the endoscope and the imaging system comprises a separate OPA at the head of the endoscope, and the OPA and the separate OPA are formed on different chips.

16. The endoscopic ablation system of claim 1, wherein the AOPA comprises an OPA in the head of the endoscope and the imaging system comprises a separate OPA at the head of the endoscope, and the OPA and the separate OPA are formed on a common integrated substrate or two chips bonded to a common chip.

17. The endoscopic ablation system of claim 1, wherein the AOPA comprises an OPA in the head of the endoscope and the imaging system comprises a separate imager at the head of the endoscope, and the OPA and the separate imager are formed on different chips.

18. The endoscopic ablation system of claim 1, wherein the endoscope comprises a swallowable capsule.

19. The endoscopic ablation system of claim 18, wherein the AOPA comprises an OPA on a first substrate and the imaging system comprises a separate OPA on a second substrate separate from the first substrate.

20. The endoscopic ablation system of claim 18, wherein the AOPA comprises an OPA in on a first substrate and the imaging system comprises a separate OPA on the first substrate.

21. The endoscopic ablation system of claim 18, wherein the AOPA comprises an OPA on a first substrate and the imaging system comprises a separate imager chip on a second substrate separate from the first substrate.

22. The endoscopic ablation system of claim 18, wherein the capsule comprises one or more of: batteries, capacitors, imaging and ablation control electronics, communication electronics, AI circuitry, laser generation electronics, and optics.

23. A method comprising using the system of claim 1 to ablate tissue in a patient.
